(19) United States
(12) Patent Application Publication
(10) Pub. No.: US 2009/0312652 A1
(43) Pub. Date: Dec. 17, 2009

(54) ELECTRONIC MANOMETER FOR APPROPRIATELY ADJUSTING INTERNAL PRESSURE OF CUFF AND METHOD FOR CONTROLLING THE SAME

(75) Inventors: Kenichi Yamakoshi, Ishikawa (JP); Shinobu Tanaka, Ishikawa (JP); Masamichi Nogawa, Ishikawa (JP); Takehiro Yamakoshi, Ishikawa (JP); Yukiya Sawanoi, Nara (JP)

Correspondence Address: MORRISON & FOERSTER LLP, 1650 TYSONS BOULEVARD, SUITE 400, MCLEAN, VA, 22102, US

(73) Assignee: Omron Healthcare Co., Ltd., Kyoto-shi (JP)

(21) Appl. No.: 12/307,116

(22) PCT Filed: Jul. 20, 2007
(86) PCT No.: PCT/JP2007/064324

§ 371 Date: Jun. 10, 2009

(30) Foreign Application Priority Data
Aug. 3, 2006 (JP) 2006-212090

(51) Int. Cl. A61B 5/0225

(52) U.S. Cl. 600/493

(57) ABSTRACT

An electronic manometer detects pressure within a cuff at which an amplitude of change in a volume of an artery of a measurement subject becomes a maximum by detecting volume of an artery of a measurement subject while superimposing a vibration of high frequency during a period of raising the pressure to apply on the cuff or during a period of lowering after raising to greater than or equal to a systolic blood pressure of the measurement subject. The electronic manometer controls the pressure to apply on the cuff so that the volume of the artery of the measurement subject becomes constant, and measures the blood pressure of the measurement subject from an increased or decreased value of the pressure to apply.

This application is a National Stage application of PCT/JP2007/064324, filed Jul. 20, 2007, which claims the benefit of priority of Japanese Application No. 2006-212090, filed Aug. 3, 2006, the entire contents of these applications hereby incorporated by reference.

### TECHNICAL FIELD

The present invention relates to an electronic manometer and a method for controlling the same, in particular, to an electronic manometer for measuring blood pressure using a volume compensation method, and a method for controlling the same.

### BACKGROUND ART

Blood pressure is one index for analyzing cardiovascular disorder, where performing risk analysis based on the blood pressure is effective in preventing cardiovascular diseases such as stroke, cardiac arrest, and cardiac infarction. In particular, morning hypertension in which the blood pressure rises in early morning is related to heart disease, stroke, and the like. Furthermore, it is known that in the morning hypertension, a symptom in which the blood pressure rapidly rises from one hour to about one and a half hour after wakeup called morning surge has a causal association with stroke. Understanding the mutual relationship between time (lifestyle) and change in blood pressure is useful in risk analysis of the cardiovascular disease. Therefore, continuous measurement of the blood pressure over a long period of time is necessary.

In monitoring patients during surgery and after surgery, checking medicinal effects in treatment with antihypertensive drug, and the like, it is very important to continuously measure the blood pressure for every heart beat and to monitor the change in blood pressure.

The pulse wave waveform for every heart beat includes information having an extremely wide usage range in a medical field such as progress of arterial sclerosis and diagnosis of heart function, and it is also important to continuously record a fluctuation in a pulse wave waveform.

In a process of wrapping a cuff at a measurement site, pressurizing the pressure within the cuff to higher than a systolic blood pressure, and then gradually depressurizing the pressure within the cuff, the conventional manometer detects the pulsation generated in the artery with a pressure sensor via the cuff, and applies a predetermined algorithm to the pressure within the cuff and the magnitude of the pulsation at the time (pulse wave amplitude) to determine the systolic blood pressure and the diastolic blood pressure (oscillometric method). Such manometer utilizes a characteristic that the pulse wave amplitude rapidly increases when the internal pressure of the cuff is near the systolic blood pressure and rapidly decreases when near the diastolic blood pressure.

In the conventional manometer, a blood flow sound (Korotkov's sound) that appears or disappears depending on the change in blood flow during pressurization and depressurization of the internal pressure of the cuff is detected with a Korotkov's sound detector such as a microphone to determine the systolic blood pressure and the diastolic blood pressure (Korotkov's sound method). Such manometer utilizes the characteristic that the Korotkov's sound appears when the internal pressure of the cuff is at the systolic blood pressure, and disappears (or muffles) near the diastolic blood pressure.

In such blood pressure measurement method, the internal pressure of the cuff needs to be gradually depressurized to determine the systolic blood pressure and the diastolic blood pressure, and thus the measurement time for one measurement requires about thirty seconds at the fastest, and it is impossible to measure the blood pressure for every heart beat.

A method of measuring the blood pressure for every heart beat includes a method of inserting a catheter into the artery, and directly measuring the intra-arterial pressure by a pressure sensor connected to the catheter (direct method). The direct method inserts the catheter into the artery, and thus needs to be conducted under a supervision of a doctor, and also provides psychological and/or physical pain such as pain and/or risk of infection to the measurement subject (patient) and can be performed only under a specified environment such as during a surgery or after a surgery.

Under such situation, a technique of noninvasively and continuously measuring the blood pressure for every heart beat has been developed.

For such a technique, for example, a blood pressure measurement method called a tonometric method is known. Here, when the superficial artery such as a radial artery is compressed flat from the body surface, the pressure on the plane can be detected by the pressure sensor to measure the blood pressure using the principle that the pressure on the plane matches the intra-arterial pressure. Actually, it is impossible to compress only the artery, and the tissues of the body surface are also simultaneously compressed, and thus the pressure detected by the pressure sensor does not necessarily match the intra-arterial pressure. Therefore, in measuring the blood pressure through the tonometric method, correction needs to be made by the blood pressure value detected through other methods (e.g., oscillometric method). Furthermore, the sensor needs to be correctly arranged on the artery to flatly compress the artery, and thus it is very troublesome to handle, and is used only in a limited state such as during a surgery or when treating in an intensive case after the surgery.

A blood pressure measurement by volume compensation method is developed as disclosed in Japanese Unexamined Patent Publication No. 54-50175 (Patent Document 1) as a method for easy and convenient measurement.

The volume compensation method is a method of compressing the artery by means of the cuff from outside the body, equilibrating the compressed pressure (cuff pressure) and the intra-arterial pressure, that is, blood pressure by maintaining the volume per unit length of the pulsating artery constant, and detecting the cuff pressure when such state is maintained to obtain a continuous blood pressure value. Since the arterial wall needs to be maintained in a no-load state (i.e., natural state not applied with pressure), two points of detecting that the artery is in the no-load state (detection of servo target value) and maintaining the relevant state (servo control) become important. In particular, determination of the servo target value directly influences the blood pressure measurement accuracy, and thus its determination is very important.

As one example of a technique related to the determination of the servo target value in the electronic manometer, Japanese Unexamined Patent Publication No. 59-156325 (Patent Document 2) discloses a technique of gradually compressing the artery with the cuff, and detecting the artery volume maximum point obtained at the relevant time to obtain the servo target value (control target value). The mode of determination of the control target value disclosed in the publication will be described with reference to FIG. 7. In FIG. 7, a cuff pressure is indicated as Pc, an output voltage (volume signal of the artery) of a photo-transistor is indicated as PGdc, and a pulse wave component of the volume signal is indicated as PGac.

In the technique disclosed in Patent Document 2, while gradually pressurizing the cuff (3 to 4 mmHg/sec), the pulse wave component of the volume signal at the relevant time is acquired, and the maximum value thereof is detected. The internal pressure of the cuff at the point the maximum value is detected is set as the servo target value.

[Patent Document 1] Japanese Unexamined Patent Publication No. 54-50175

[Patent Document 2] Japanese Unexamined Patent Publication No. 59-156325

### DISCLOSURE OF THE INVENTION

### Problems to be Solved by the Invention

In the technique disclosed in Patent Document 2, an accurate servo target value (control target value) can be detected, but 30 seconds or more are required at minimum to detect the servo target value. Thus, 30+α seconds or more are required until a first blood pressure determination from a start of measurement, and a measurement subject feels inconvenience.

During continuous blood pressure measurement, the servo target value sometimes fluctuates due to blood pressure fluctuation, stress, change in environment, and the like, and the servo target value needs to be redetected each time, but in this case as well, at least 30 seconds are required until the determination of the servo target value. Thus, the blood pressure measurement needs to be interrupted, and the control target value needs to be re-determined, whereby the blood pressure fluctuation during the continuous blood pressure measurement cannot be rapidly responded.

In view of the above situations, it is an object of the present invention to determine a control target value in a short period of time in an electronic manometer for measuring the blood pressure using a volume compensation method.

### Means for Solving the Problems

An electronic manometer of the present invention includes a cuff attached to a blood pressure measurement site of a measurement subject; a pressure adjustment unit for adjusting a pressure to apply on the cuff; a pressure detection unit for detecting a pressure within the cuff; an artery volume detection unit for detecting a volume of an artery of the measurement subject, which changes by a change in the pressure within the cuff and pulsation; and a measurement unit for controlling the volume of the artery of the measurement subject so as to be constant by causing the pressure adjustment unit to increase or decrease the pressure to apply on the cuff based on the change in the volume of the artery of the measurement subject, and measuring a blood pressure of the measurement subject from an increased or decreased value of the pressure to apply on the cuff; wherein the measurement unit includes a pressure maintaining unit for detecting the pressure within the cuff at which an amplitude of the change in the volume of the artery of the measurement subject becomes a maximum, and maintaining the pressure, a feedback control unit for operating the pressure adjustment unit based on the change in the volume of the artery of the measurement subject and feedback controlling so that the amplitude of the change in the volume of the artery of the measurement subject becomes a minimum, and an extracting unit for extracting the pressure within the cuff when the amplitude of the change in the volume of the artery of the measurement subject becomes a minimum by the control from the feedback control unit as the blood pressure of the measurement subject; and the pressure maintaining unit causes the artery volume detection unit to detect the volume of the artery of the measurement subject while superimposing a vibration of high frequency during a period of causing the pressure adjustment unit to raise the pressure to apply on the cuff or during a period to lower after raising to greater than or equal to a systolic blood pressure of the measurement subject to detect the pressure within the cuff at which the amplitude of the change in the volume of the artery of the measurement subject becomes a maximum.

In the electronic manometer of the present invention, the pressure maintaining unit detects the pressure within the cuff at which the amplitude of the change in the volume of the artery of the measurement subject becomes a maximum at a start of measurement of the blood pressure by the measurement unit and at a predetermined time other than at the start of measurement.

Preferably, the predetermined time is when the blood pressure measured by the measurement unit exceeds a predetermined value or becomes lower than a specific value.

Preferably, the predetermined time is when an amount of change in the volume of the artery of the measurement subject detected by the artery volume detection unit exceeds a constant value during the measurement of the blood pressure by the measurement unit.

Preferably, the predetermined time is when the amount of change in the pressure within the cuff detected by the pressure detection unit exceeds a certain value during the measurement of the blood pressure by the measurement unit.

An operation unit for accepting an operation from outside is further preferably arranged; and the predetermined time is when the operation is made to the operation unit.

The pressure maintaining unit preferably detects the pressure within the cuff at which the amplitude of the change in the volume of the artery of the measurement subject becomes a maximum based on a detection result of the volume of the artery of the measurement subject by the artery volume detection unit in a period the pressure adjustment unit adjusts the pressure to apply on the cuff by one heart beat based on the control by the feedback control unit.

A method of controlling an electronic manometer of the present invention is a method of controlling an electronic manometer including a cuff attached to a blood pressure measurement site of a measurement subject, the method causing the electronic manometer to execute the steps of detecting a pressure within the cuff at which an amplitude of change in a volume of an artery of the measurement subject becomes a maximum; maintaining the detected pressure within the cuff; adjusting a pressure to apply on the cuff based on the change in the volume of the artery of the measurement subject while performing a feedback control so that the amplitude of the change in the volume of the artery of the measurement subject becomes a minimum; and extracting the pressure within the cuff when the amplitude of the change in the volume of the artery of the measurement subject becomes a minimum by the feedback control as the blood pressure of the measurement subject; wherein the step of detecting the pressure within the cuff is a step of detecting the volume of the artery of the measurement subject while superimposing a vibration of high frequency during a period of raising the pressure to apply on the cuff or during a period of lowering after raising the pressure to apply on the cuff to greater than or equal to a systolic blood pressure of the measurement subject to detect the pressure within the cuff at which the amplitude of the change in the volume of the artery of the measurement subject becomes a maximum. In the method of controlling the electronic manometer of the present invention, the step of detecting the pressure within the cuff is executed at a start of blood pressure measurement in the electronic manometer and at a predetermined time other than at the start of measurement.

According to the present invention, the pressure within the cuff when the amplitude of the change in the volume of the artery of the measurement subject detected with the vibration of high frequency superimposed on the pressure to apply on the cuff becomes a maximum is the pressure corresponding to the target value (control target value) to which the pressure within the cuff is to be maintained. According to the present invention, the pressure within the cuff at which the amplitude of the change in the volume of the artery of the measurement subject becomes a maximum is detected at the start of measurement of blood pressure by the measurement unit and at a predetermined time other than at the start of measurement.

The pressure within the cuff when the change in the volume of the artery of the measurement subject becomes a maximum then can be obtained for every feedback control corresponding to one heart beat. The servo target value due to blood pressure fluctuation, stress, change in environment and the like during continuous blood pressure measurement can also respond to fluctuation.

### BRIEF DESCRIPTION OF THE DRAWINGS

FIG. 1 is an overview diagram of an electronic manometer according to one embodiment of the present invention.

FIG. 2 is a view schematically showing a hardware configuration of the electronic manometer of FIG. 1.

FIG. 3 is a flowchart of a process executed in the electronic manometer shown in FIG. 1 when a power supply is turned ON to measure blood pressure.

FIG. 4 is a flowchart of a sub-routine for detecting a control target value in a process shown in FIG. 3.

FIG. 5 is a view showing data handled in a CPU of the electronic manometer shown in FIG. 1.

FIG. 6 is a flowchart of a variant of the process shown in FIG. 3.

FIG. 7 is a view for describing a mode of determining the control target value in a conventional electronic manometer.

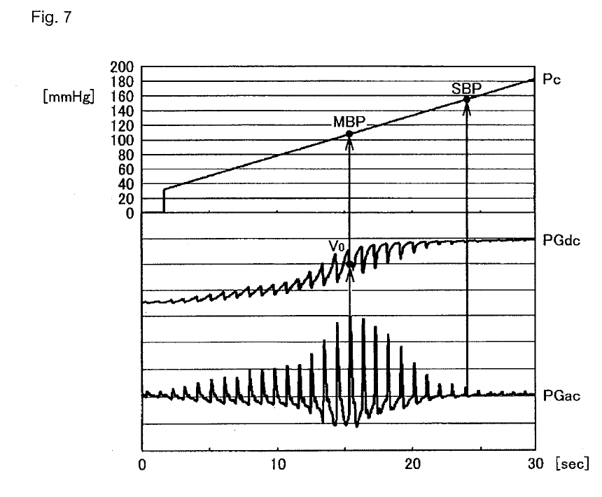

### DESCRIPTION OF REFERENCE SYMBOLS

1 Electronic manometer
2 Cuff
3 Tube
4 Display unit
5 Operation unit
6 Timer
10 CPU
11, 12 Memory
13 Oscillation circuit
14 Pump drive circuit
15 Valve drive circuit
16 Artery volume detection circuit
20 Air bag
21 Artery volume sensor
30 Power supply
31 Pressure sensor
32 Pump
33 Valve
51 Power switch
52 Measurement switch
53 Stop switch
54 Record call-out switch

### BEST MODE FOR CARRYING OUT THE INVENTION

An embodiment of an electronic manometer according to the present invention will be described below with reference to the drawings.

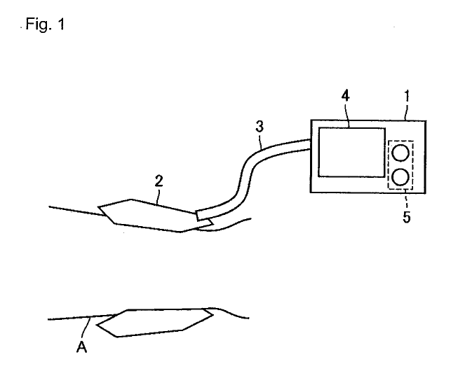

FIG. 1 is an overview diagram of an electronic manometer according to one embodiment of the present invention.

The electronic manometer 1 includes a cuff 2, and measures blood pressure of a measurement subject with the cuff 2 wrapped around a wrist A of the measurement subject. In the electronic manometer according to the present invention, a measurement site of the blood pressure is not limited to the wrist. The measurement sites may be a fingertip, an upper arm, and the like as long as a volume of an artery can be detected, as hereinafter described.

The electronic manometer 1 is arranged on a front surface with a display unit 4 and an operation unit 5 with a plurality of operation buttons. The electronic manometer 1 also includes a tube 3 for connecting a built-in pump (later-described pump 32) and the cuff 2. The cuff 2 internally includes an air bag.

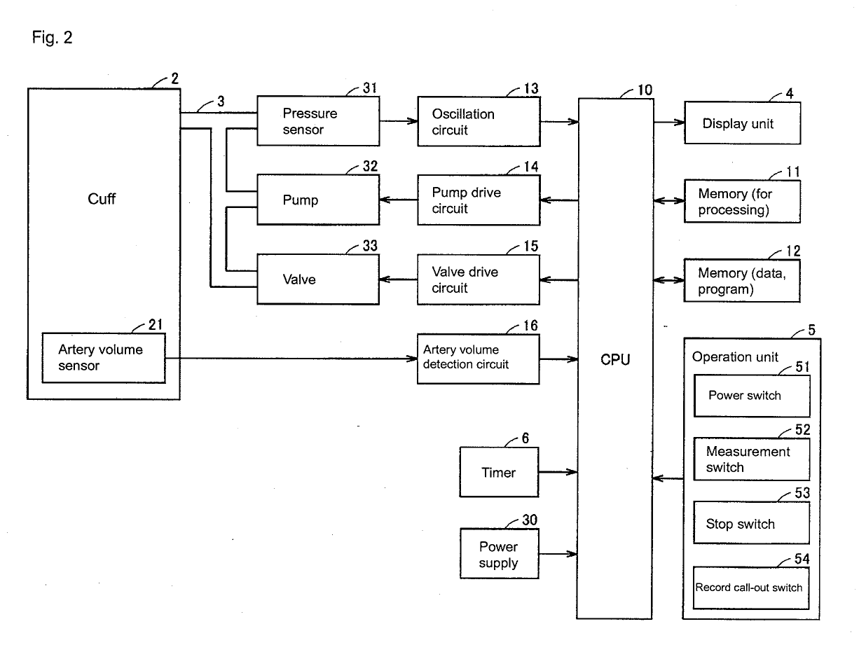

FIG. 2 is a view schematically showing a hardware configuration of the electronic manometer 1.

The electronic manometer 1 includes, in addition to the display unit 4 and the operation unit 5 discussed above, a CPU (Central Processing Unit) 10 for wholly controlling the operation of the electronic manometer 1, a memory 11 functioning as a work memory of the CPU 10, a memory 12 for storing various information (program and data), a timer 6, and a power supply 30 for supplying power to the CPU 10. The operation unit 5 includes a power switch 51 operated to switch ON/OFF of the power supply with respect to the electronic manometer 1, a measurement switch 52 operated to cause the electronic manometer 1 to start the measurement of the blood pressure, a stop switch 53 operated to cause the electronic manometer 1 to stop the measurement operation, and a record call-out switch 54 operated to call out the measurement result of the blood pressure and the like stored in the memory 12.

The electronic manometer 1 further includes, in addition to the cuff 2 and the tube 3 described above, a pressure sensor 31 for measuring the pressure of the air bag of the cuff 2, an oscillation circuit 13 for converting an output from the pressure sensor 31 to frequency and inputting the same to the CPU 10, a pump 32 for supplying air to the air bag of the cuff 2, a pump drive circuit 14 for driving the pump 32, a valve 33 for opening/closing a connecting portion of the tube 3 and the pump 32, and a valve drive circuit 15 for driving the valve 33. In the electronic manometer 1, the CPU 10 controls the operation of the pump drive circuit 14 and the valve drive circuit 15 to control the pressure of the air bag of the cuff 2.

The electronic manometer 1 further includes an artery volume sensor 21 attached to the cuff 2, and an artery volume detection circuit 16 input with a detection output of the artery volume sensor 21. The artery volume sensor 21 is a sensor for detecting the volume of the artery at the blood pressure measurement site of the measurement subject, and is configured by a photoelectric sensor and the like. The photoelectric sensor specifically includes a light emission diode, and a phototransistor arranged so as to face the light emission diode with the measurement site in between. The artery volume detection circuit 16 controls a light emission quantity of the light emission diode, and is input with a light receiving quantity of the phototransistor. Thus, in the artery volume detection circuit 16, the volume of the artery is detected based on a transmitted light quantity reaching the photodiode of the light emitted by the light emission diode, which is the light of absorption band of hemoglobin contained in the blood (red blood cell) flowing through the blood vessel. In the electronic manometer according to the present invention, the volume of the artery may be detected, other than by the photoelectric sensor, using existing methods such as an impedance plethysmograph.

In the electronic manometer 1, the blood pressure (systolic blood pressure, diastolic blood pressure) is detected using a volume compensation method. The blood pressure measurement through the volume compensation method obtains the blood pressure by applying external pressure to the artery from ex vivo, performing control to constantly equilibrate with the in vitro pressure (intra-arterial pressure, that is, blood pressure), maintaining the arterial wall in a no-load state, and measuring the in vitro pressure at that point. The blood pressure measurement using the volume compensation method is specifically described in Patent Document 1.

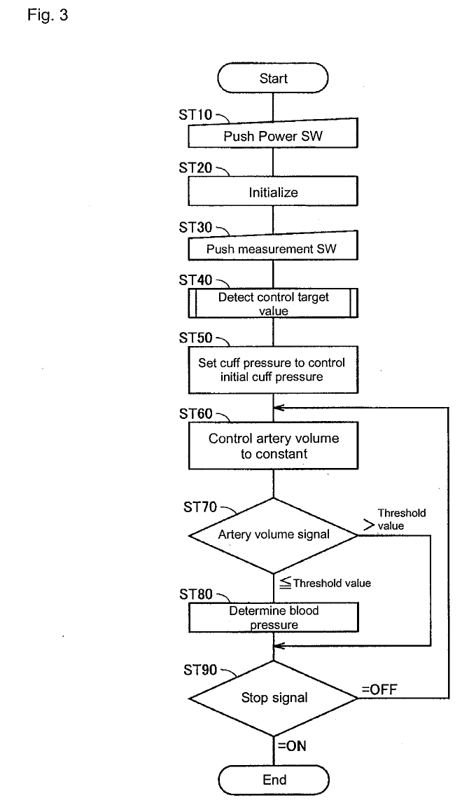

FIG. 3 is a flowchart of a process executed in the electronic manometer 1 when the power supply is turned ON to measure the blood pressure.

With reference to FIG. 3, first the power switch 51 is pushed by the measurement subject or a user in step ST10. Initialization is then performed in the electronic manometer 1 in step ST20. The initialization includes initialization of the memory 11, exhaust of air in the cuff 2, and correction of 0 mmHg of the pressure sensor 31 by the CPU 10.

When the measurement switch 52 is pushed (step ST30) after initialization, a control target value for the no-load state of the cuff 2 is detected in step ST40 in the electronic manometer 1. The processing content for detecting the control target value in step ST40 will be hereinafter described.

When the control target value is determined, the CPU 10 adjusts the pressure of the air bag of the cuff 2 to a control initial cuff pressure, to be hereinafter described, (step ST50), and then determines the blood pressure of the measurement subject while feedback controlling the pressure of the air bag of the cuff 2 so that the amplitude of an artery volume signal becomes a minimum (steps ST60 to ST80). The artery volume signal is a signal output from the artery volume detection circuit 16 to the CPU 10, and is a signal corresponding to the volume of the artery of the measurement subject.

In step ST60, the CPU 10 controls the pressure of the air bag of the cuff 2 so that the amplitude of the artery volume signal becomes a minimum based on the detection output of the artery volume detection circuit 16. When referring to “so that the amplitude of the artery volume signal becomes a minimum”, it means so that the volume of the artery of the measurement subject becomes lower than or equal to a threshold value defined in advance. For instance, if the detection output of the artery volume detection circuit 16 infers that the volume of the artery of the measurement subject exceeds the threshold value, the CPU 2 controls the operation of the valve 33 so as to raise the pressure of the air bag of the cuff 2 according to the exceeding degree. In the present embodiment, when measuring the blood pressure, the pump 32 is constantly operated, and the pressure of the air bag of the cuff 2 is controlled by the opening/closing mode of the valve 33. The pressure of the air bag of the cuff 2 may be controlled by the driving mode of the pump 32 with the valve 33 constantly opened, or may be controlled by combining both the opening/closing mode of the valve 33 and the driving mode of the pump 32.

In step ST70, the CPU 10 determines whether or not the artery volume signal infers that the volume of the artery of the measurement subject is lower than or equal to the threshold value. If determining as lower than or equal to the threshold value, the CPU 10 decides the pressure of the air bag of the cuff 2 at the relevant point as the blood pressure of the measurement subject in step ST80, and proceeds the process to step ST90. If determining as exceeding the threshold value, the CPU 10 directly proceeds the process to step ST90 without executing the process of step ST80. In step ST80, the CPU 10 displays the determined blood pressure value on the display unit 4, or stores the same in the memory 12 (e.g., along with time at the relevant point).

In step ST90, the CPU 10 determines whether or not the stop signal is turned ON, and returns the process to step ST60 if determining as turned OFF and terminates the measurement if determining as turned ON. The stop signal is a signal for stopping the measurement of the blood pressure, and is turned OFF at the initialization of step ST20, and is turned ON when the stop switch 53 is pushed or after elapse of a predetermined time from the start of measurement of the blood pressure (pushing of measurement switch 52 in step ST30).

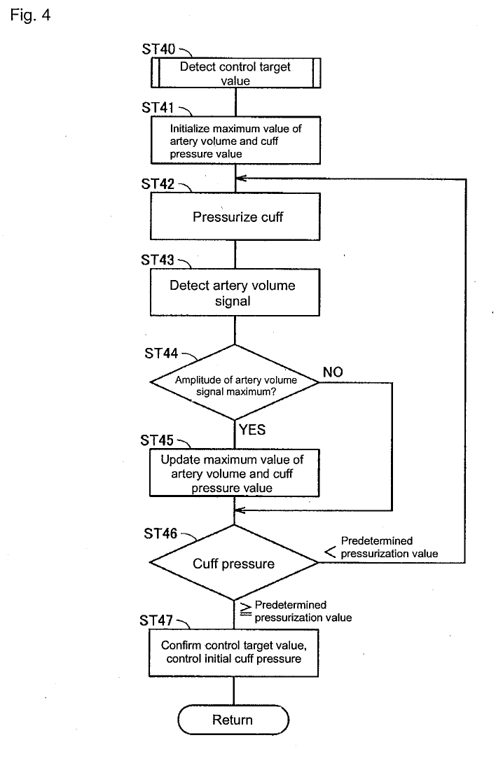

FIG. 4 is a flowchart of a sub-routine for detecting the control target value in step ST40.

With reference to FIG. 4, in the detection process of the control target value, the CPU 10 first initializes a maximum value of the artery volume (artery volume signal) stored in the memory 12 and a pressure value of the air bag of the cuff 2 at the point the maximum value is detected in step ST41.

In step ST42, the CPU 10 then pressurizes the air bag of the cuff 2. The pressurization of the air bag of the cuff 2 is performed not by simply raising the pressure linearly, but by superimposing a high frequency minimal pressure vibration (e.g., frequency of 20 Hz, amplitude of 10 mmHg) on a linear pressure raising waveform. The vibration of high frequency is superimposed on the pressure to apply on the cuff 2.

The CPU 10 detects the artery volume signal from the artery volume detection circuit 16 in step ST43, and determines whether or not the amplitude of the artery volume signal at the relevant point corresponds to the maximum value of an amount of change in the artery volume in step ST44. The artery volume signal is obtained as high frequency component of the data output from the artery volume sensor 21, as hereinafter described. The determination on whether corresponding to the maximum value or not in step ST44 is made by whether the amount of change (e.g., derivative value) in the amplitude of the artery volume signal immediately before is a positive value, and the amount of change in the amplitude of the artery volume signal at the relevant point is a negative value.

The CPU 10 proceeds the process to step ST45 when determining as corresponding to the maximum value in step ST44, and proceeds the process to step ST46 when determining as not corresponding to the maximum value.

In step ST45, the CPU 10 stores the maximum value of the artery volume signal (its amplitude) at the relevant point and the pressure value of the air bag of the cuff 2 at the relevant point in the memory 12, and proceeds the process to step ST46. The maximum value of the amplitude of the artery volume signal stored here is handled as the control target value in the feedback control in the electronic manometer 1. If values are already stored in the memory 12, the values are updated to the most recent values.

In step ST46, the CPU 10 determines whether or not the pressure of the air bag of the cuff 2 has reached a predetermined pressure, and proceeds the process to step ST46 when determining as reached and returns the process to step ST42 when determining as not reached.

In step ST47, the CPU 10 confirms the maximum value of the amplitude of the artery volume signal stored in the memory 12 at the relevant point and the pressure value of the air bag of the cuff 2 at the relevant point as the control target value and the control initial cuff pressure, respectively, and returns the process to step ST40 of FIG. 3.

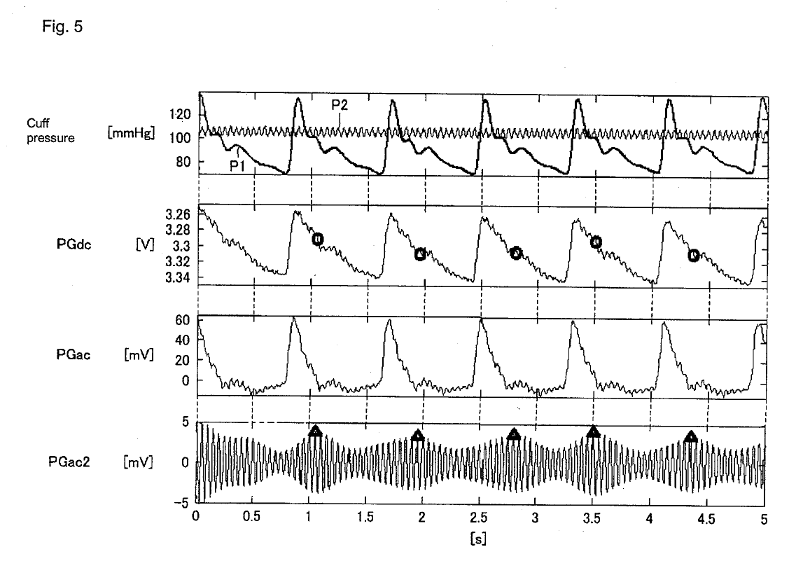

The detection of the control target value in the present embodiment described above will be specifically described with reference to FIG. 5. FIG. 5 is a view showing data handled in the CPU 10 of the electronic manometer 1. Specifically, FIG. 5 shows the pressure to apply on the air bag of the cuff 2 according to the feedback control as P1, the vibration of high frequency superimposed to detect the control target value as P2, the detection output of the artery volume sensor 21 as PGdc, the data removed with DC component of the PGdc as PGac, and the data processed in a band pass filter of the same frequency as the P2 with respect to the PGac as PGac2, and also shows time change of each data.

When the air bag of the cuff 2 is controlled to the pressure at which P2 is superimposed on P1, the amplitude of the PGac2 takes a maximum value (maximal value) at the point the pressure of the air bag of the cuff 2 becomes equilibrium with the intra-arterial pressure. In FIG. 5, triangle mark is given to the maximum value for every heart beat of the PGac2.

In the present embodiment, the control target value stored in the memory 12 is the PGdc (O mark in FIG. 5) at the point the amplitude of the PGac2 takes the maximum value (maximal value). In the present embodiment, the pressure value of the air bag of the cuff 2 at the relevant point is also stored in the memory 12.

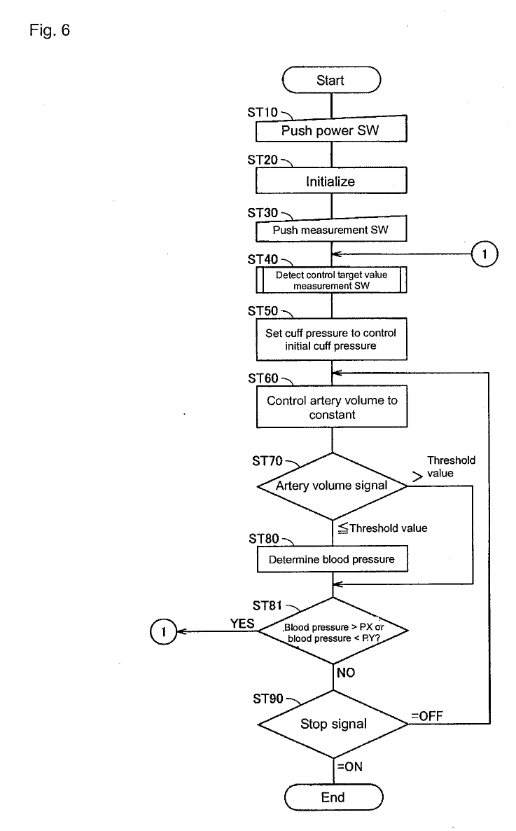

In the embodiment described above, the control target value and the pressure value are detected at the start of measurement of the blood pressure in the electronic manometer 1. The timing of detecting such values in the electronic manometer according to the present invention is not limited to the start of measurement of the blood pressure. For instance, as shown in FIG. 6, whether or not outside a predetermined range (i.e., exceeding a predetermined value PX, or below a specific value PY) is determined for the blood pressure value decided in step ST80 (step ST81), and detection may be made when outside the range (determined as YES in step ST81).

Detection may be made when turning ON the power with respect to the electronic manometer 1, detection may be made when a dedicated switch is arranged on the operation unit 5 and such switch is operated, detection may be made when the pressure of the air bag is suddenly changed during the blood pressure measurement (i.e., when an amount of change in pressure exceeds a certain value defined in advance), or the volume of the artery of the measurement subject detected in the artery volume detection circuit 16 continuously stored in the memory 12 etc., and detection may be made when the value of the artery volume is changed exceeding a constant value defined in advance with respect to the stored value.

In the embodiment described above, the control target value and the pressure value are detected within a period of raising the pressure of the air bag of the cuff 2 (step ST42) in the flowchart shown in FIG. 4. In the example described with reference to FIG. 5, the control target value and the pressure value are detected within a period of lowering the pressure of the air bag of the cuff 2. Thus, in the present invention, the control target value and the pressure value may be detected within the period of raising or within the period of lowering the pressure of the air bag of the cuff 2. However, when detecting within the period of lowering, it at least needs to be a period of lowering after raising the pressure of the air bag of the cuff 2 to greater than or equal to a systolic blood pressure of the measurement subject. To this end, the systolic blood pressure of the measurement subject measured up to now (or input by the measurement subject by operating the operation unit 5) is stored in the memory 12, and the control target value and the pressure value are preferably detected after raising the pressure of the air bag of the cuff 2 to greater than or equal to such blood pressure.

The control target value and the pressure value may be detected with the pressure value of the air bag of the cuff 2 controlled to the pressure value detected before and stored in the memory 12, and with the vibration of high frequency superimposed on the control waveform of the pressure.

As shown in FIG. 4 or FIG. 5, after detecting the control target value and the pressure value with the pressure of the air bag of the cuff 2 raised or lowered while being superimposed with the vibration of high frequency, the control target value and the pressure value are again detected with the pressure of the air bag of the cuff 2 controlled with the detected pressure value and with the pressure controlled so as to superimpose the vibration of high frequency, and the values detected afterward may be stored in the memory 12. The values detected first or afterward may be stored in the memory 12 assuming the detection is successful only when the difference between the values detected first and the values detected afterward is within a predefined range.

As shown in FIG. 5, in the electronic manometer 1, the maximum value of the amplitude of the artery volume signal can be detected for every heart beat of the measurement subject in the artery volume signal. The control target value and the pressure value may be detected for one heart beat, that is, at the point one maximum value of the amplitude of the artery volume signal is obtained, or may be detected by taking an average of each value for a plurality of heart beats.

Each embodiment disclosed herein is illustrative in all aspects, and should not be construed as being restrictive. The scope of the present invention is defined by the claims rather than by the description given above, and is intended to encompass all meanings equivalent to the claims and all modifications within the scope.

1. An electronic manometer comprising:
a cuff attached to a blood pressure measurement site of a measurement subject;
a pressure adjustment unit for adjusting a pressure to apply on the cuff;
a pressure detection unit for detecting a pressure within the cuff;
an artery volume detection unit for detecting a volume of an artery of the measurement subject, which changes by a change in the pressure within the cuff and pulsation; and
a measurement unit for controlling the volume of the artery of the measurement subject so as to be constant by causing the pressure adjustment unit to increase or decrease the pressure to apply on the cuff based on the change in the volume of the artery of the measurement subject, and measuring a blood pressure of the measurement subject from an increased or decreased value of the pressure to apply on the cuff; wherein
the measurement unit includes,
a pressure maintaining unit for detecting the pressure within the cuff at which an amplitude of the change in the volume of the artery of the measurement subject becomes a maximum, and maintaining the pressure,
a feedback control unit for operating the pressure adjustment unit based on the change in the volume of the artery of the measurement subject and feedback controlling so that the amplitude of the change in the volume of the artery of the measurement subject becomes a minimum, and
an extracting unit for extracting the pressure within the cuff when the amplitude of the change in the volume of the artery of the measurement subject becomes a minimum by the control from the feedback control unit as the blood pressure of the measurement subject;
the pressure maintaining unit,
causes the artery volume detection unit to detect the volume of the artery of the measurement subject while superimposing a vibration of high frequency during a period of causing the pressure adjustment unit to raise the pressure to apply on the cuff or during a period to lower after raising to greater than or equal to a systolic blood pressure of the measurement subject to detect the pressure within the cuff at which the amplitude of the change in the volume of the artery of the measurement subject becomes a maximum, and
detects the pressure within the cuff at which the amplitude of the change in the volume of the artery of the measurement subject becomes a maximum at a start of measurement of the blood pressure by the measurement unit and at a predetermined time other than at the start of measurement.

2. (canceled)

3. The electronic manometer according to claim 1, wherein the predetermined time is when the blood pressure measured by the measurement unit exceeds a predetermined value or becomes lower than a specific value.

4. The electronic manometer according to claim 1, wherein the predetermined time is when an amount of change in the volume of the artery of the measurement subject detected by the artery volume detection unit exceeds a constant value during the measurement of the blood pressure by the measurement unit.

5. The electronic manometer according to claim 1, wherein the predetermined time is when an amount of change in the pressure within the cuff detected by the pressure detection unit exceeds a certain value during the measurement of the blood pressure by the measurement unit.

6. The electronic manometer according to claim 1, further comprising an operation unit for accepting an operation from outside; wherein
the predetermined time is when the operation is made to the operation unit.

7. The electronic manometer according to claim 1, wherein the pressure maintaining unit detects the pressure within the cuff at which the amplitude of the change in the volume of the artery of the measurement subject becomes a maximum based on a detection result of the volume of the artery of the measurement subject by the artery volume detection unit in a period the pressure adjustment unit adjusts the pressure to apply on the cuff by one heart beat based on the control by the feedback control unit.

8. A method of controlling an electronic manometer including a cuff attached to a blood pressure measurement site of a measurement subject, the method causing the electronic manometer to execute the steps of:
detecting a pressure within the cuff at which an amplitude of change in a volume of an artery of the measurement subject becomes a maximum;
maintaining the detected pressure within the cuff;
adjusting a pressure to apply on the cuff based on the change in the volume of the artery of the measurement subject while performing a feedback control so that the amplitude of the change in the volume of the artery of the measurement subject becomes a minimum; and
extracting the pressure within the cuff when the amplitude of the change in the volume of the artery of the measurement subject becomes a minimum by the feedback control as the blood pressure of the measurement subject; wherein
the step of detecting the pressure within the cuff is executed at a start of blood pressure measurement in the electronic manometer and at a predetermined time other than at the start of measurement, and is a step of detecting the volume of the artery of the measurement subject while superimposing a vibration of high frequency during a period of raising the pressure to apply on the cuff or during a period of lowering after raising the pressure to apply on the cuff to greater than or equal to a systolic blood pressure of the measurement subject to detect the pressure within the cuff at which the amplitude of the change in the volume of the artery of the measurement subject becomes a maximum.
